(19) United States
(12) Patent Application Publication
(10) Pub. No.: US 2008/0038685 A1
(43) Pub. Date: Feb. 14, 2008

(54) Light Irradiation Device For Oral Cavity

(75) Inventors: Hidenari Sakaguchi, Kanagawa-ken (JP); Satoshi Nakamura, Tokyo (JP)

Correspondence Address: GREENBLUM & BERNSTEIN, P.L.C., 1950 ROLAND CLARKE PLACE, RESTON, VA, 20191, US

(73) Assignee: SHOWA YAKUHIN KAKO CO., LTD., Tokyo (JP)

(21) Appl. No.: 10/596,430

(22) PCT Filed: Dec. 15, 2004
(86) PCT No.: PCT/JP04/19150

§ 371 Date: May 14, 2007

(30) Foreign Application Priority Data
Dec. 15, 2003 (JP) 2003-416309

(51) Int. Cl. A61B 1/24

(52) U.S. Cl. 433/29

(57) ABSTRACT

An object to be solved by the present invention is to provide means for uniformly irradiating oral cavity in a simple and safe manner. The present invention provides an intraoral light irradiation device, wherein an electroluminescence (EL) element is used.

### TECHNICAL FIELD

The present invention relates to an intraoral light irradiation device, for example, an intraoral light irradiation device using a mouthpiece with an intrinsic EL.

### BACKGROUND ART

In general, a mouthpiece, a mouthguard, or what can be referred to as a “night form preguard has been used for protection of the oral cavity during dental treatment or hard exercise such as boxing, prevention of bruxism during sleep, and the like. In addition, JP Patent Publication (Kokai) No. 6-217996 (1994) discloses a method of forming a mouthguard for toothbrushing, to the inside surface of which toothpaste is applied, which is characterized in that plasticity is imparted via heating to a mouthguard for toothbrushing made of thermoplastic resin, a groove is formed on the mouthguard so as to correspond to a tooth form, and toothpaste is applied inside the groove, such that the mouthguard can impart toothpaste to the gum.

Meanwhile, improved esthetics relating to teeth have been strongly desired in dental treatment, and thus teeth bleaching has been performed. Tooth discoloration or staining occurs due to deposition of chromatic substances contained in tobaccos or favorite foods such as coffee and multiplication of pigment-producing bacteria. Also, tooth discoloration occurs due to external factors such as influences of metallic materials in the oral cavity, or the like. In addition, tooth discoloration occurs due to internal factors such as aging, metabolic disorders, inheritance, and dental disorders. To cope with such discoloration derived from external or internal factors, a method for bleaching teeth using photocatalyst, titanium oxide, a peroxide such as hydrogen peroxide or urea peroxide, a reductant, an acid, or an alkali has been employed. To promote bleaching effects obtained by such agents, heating or light irradiation may be carried out in combination. Further, light irradiation is often performed in the oral cavity when, for example, eliminating bacteria in the oral cavity, curing resin used in dental caries treatment, and activating titania. Currently, light sources such as xenon, halogen and laser are used as light irradiation means. However, the light irradiation means generates heat in parallel with light irradiation, which causes damage on biological body/tooth, and the damage is particularly increased when laser is used, due to its strong power. Furthermore, the light irradiation cannot be uniformly provided to an entire tooth surface.

JP Patent Publication (Kokai) No. 2000-70292 A describes a dental light irradiation device for removing intraoral bacteria or curing a filled resin which uses a light source having a plurality of light emission diodes which is characterized in that it includes: a mouthpiece having an recess for inserting a detention and sandwiching the dentition from the back side and the front side thereof; a plurality of light emission diodes which is arranged on the inner side of the recess of the mouthpiece with the light emission surfaces being facing to the dentition; and a switch for turning on/off the light emission diodes. However, the method described in JP Patent Publication (Kokai) No. 2000-70292 A does not provide uniform light irradiation over an entire tooth surface. Also, the LEDs which are arranged lengthwise increases the thickness of the mouthpiece to about 20 mm or more, which is not practical.

### DISCLOSURE OF THE INVENTION

An object to be solved by the present invention is to provide means for uniformly irradiating an entire tooth surface in oral cavity in a simple and safe manner. Another object to be solved by the present invention is to provide means for irradiating the oral cavity with a light having a wavelength within the range of visible light without generating heat.

The inventors of the present invention eagerly studied to achieve the above objects, and found that an electroluminescence (EL) is preferred as means for irradiating oral cavity, because the electroluminescence is a thin sheet and can emit a uniform light that does not generate heat with extremely low power consumption, and the electroluminescence sheet can be cut to a different size and shape. Thus, the inventors of the present invention presumed that the oral cavity can be irradiated by a mouthpiece which is placed in the oral cavity with an EL element being applied thereto, which led to the present invention.

Thus, the present invention provides an intraoral light irradiation device, wherein an electroluminescence (EL) element is used.

Preferably, the intraoral light irradiation device which is composed of a mouthpiece having an electroluminescence (EL) element is provided.

Preferably, the electroluminescence (EL) element can emit visible light having a wavelength from 300 nm to 1000 nm. More preferably, the electroluminescence (EL) element can emit visible light. Particularly preferably, the electroluminescence (EL) element can emit blue or green visible light. Preferably, the electroluminescence (EL) element is formed in a sheet, and is more preferably formed in a sheet having a thickness of 2 mm or less. Preferably, the electroluminescence (EL) element is an organic EL.

Preferably, a display-type EL element which is formed by depositing electroluminescence (EL) on a glass substrate and illuminates upon an application of voltage, is used. Preferably, plastic is laminated on the surface of the electroluminescence (EL) element. Preferably, the mouthpiece has a concavo-convex shape which is adapted to a dentition, and the concavo-convex shape is provided with the electroluminescence (EL) element.

### BRIEF DESCRIPTION OF THE DRAWINGS

FIG. 1 shows results of a decomposition experiment of discolored tetracycline using EL light irradiation;

FIG. 2 shows results of a decomposition experiment of methylene blue by titanium oxide using EL light irradiation;

FIG. 3 shows results of a decomposition experiment of hematoporphyrin by hydrogen peroxide using EL light irradiation;

FIG. 4 shows results of a decomposition experiment of hematoporphyrin by titanium oxide using visible light-illuminated;

FIG. 5 is a schematic view showing an embodiment of a mouthpiece according to the present invention, in which reference numeral 1 designates a mouthpiece, reference numeral 2 designates a groove, reference numeral 3 designates a sidewall, reference numeral 4 designates a sidewall, and reference numeral 5 designates an edge; and

FIG. 6 is a view showing attachment examples of an EL sheet to a mouthpiece (mouth guard).

### BEST MODE FOR CARRYING OUT THE INVENTION

A method and an embodiment for practicing the present invention will be explained below.

The intraoral light irradiation device of the present invention is characterized in that it uses an electroluminescence (EL) element, and it is composed of a mouthpiece having an electroluminescence (EL) element for example. The present invention realizes a practical use of a dental light irradiation device by attaching an electroluminescence (EL) element to a mouthpiece. Alternatively, the EL itself may be molded into a simple mouthpiece shape to use.

More specifically, the present invention relates to a light irradiation device used for teeth bleaching, bacteria elimination in the oral cavity, and resin curing. Preferably, the mouthpiece used in the present invention is formed into a shape suitable for the application of the mouthpiece to the tooth form of a person or to a part thereof.

One feature of the present invention is that a mouthpiece is used to retain an electroluminescence (EL) element. Heretofore, mouthpieces have mainly been used for the purpose of protection of the oral cavity during exercise, prevention of bruxism during sleep, administration of toothpaste or a bleaching agent (when administering toothpaste or a bleaching agent, the mouthpiece is worn for a short time, about several minutes, in general), and the like. The inventors of the present invention are the first to have found that a mouthpiece can be used for the purpose of light irradiation in the oral cavity.

That is, the findings of the inventors of the present invention are novel and creative in the sense that irradiation of light generated from an electroluminescence (EL) element which have been applied to a mouthpiece, causes teeth bleaching, bacteria elimination in the oral cavity, or resin curing, each of which results in realization of expected effects.

The term “mouthpiece” used herein indicates a device that can cover teeth and/or the gum when such mouthpiece is inserted into the oral cavity. The term imparts a concept that encompasses a dental tray, a mouthguard, a night form preguard, and the like, which have been conventionally known. Such mouthpiece is not particularly limited in terms of, for example, shape, size, or material thereof, as long as it satisfies the requirements described herein. Particularly preferably, the mouthpiece does not disturb oral functions such as respiration and conversation, and is easily removable. However, the mouthpiece does not easily fall off after being applied, and can be used without causing discomfort.

The structure of the mouthpiece can be divided into a body part and a tooth form part (which comes into contact with teeth and the gum) based on function. A mouthpiece is classified as corresponding to one of the following three types, depending on the person responsible for molding the body part and the tooth form part: (1) stock type (where the body part and the tooth form part are molded by a manufacturer); (2) mouth-formed type (where the body part and the tooth form part are molded by a user); and (3) custom-made type (where the body part and the tooth form part are molded by a professional technician such as a dentist or a dental technician). A mouthpiece of the present invention may be classified as corresponding to any one of the above three types. Further, the type of a mouthpiece of the present invention may be an adequate combination of the above three types, such as a combination of a stock or custom-made type and a mouth-formed type in a manner such that the body part is molded by a manufacturer or a professional technician, and then only the tooth form part is molded by a user.

Preferably, the mouthpiece used in the present invention can be formed into a shape suitable for the application of the mouthpiece to the tooth form of a person or to a part thereof. The phrase “a shape suitable for application” indicates that the mouthpiece merely has to have a shape suitable for the application of the mouthpiece to the tooth form of the average person (for instance, when an adult is subjected to a treatment, a mouthpiece for adults, the size and shape of which correspond to the tooth form of the average adult, can be produced, and when a child is subjected to a treatment, a mouthpiece for children, the size and shape of which correspond to the tooth form of the average child, can be produced). Thus, a mouthpiece does not necessarily have an original shape that perfectly corresponds to the tooth form of a specific individual. For instance, in the case of a mouth-formed type mouthpiece, a mouthpiece formed with thermoplastic resin, which has a shape suitable for the application of the mouthpiece to the tooth form of the average person, is made. Immediately before use for a specific patient, resin of the mouthpiece is plasticized by heating (e.g., by soaking it in hot water) such that the mouthpiece is formed into a shape corresponding to the tooth form of the patient. Then, an electroluminescence (EL) element is applied to the mouthpiece, and then the mouthpiece can be applied to the patient.

Meanwhile, in the case of a stock type or custom-made type mouthpiece, a mouthpiece having a shape corresponding to the tooth form of a patient is originally made. Thus, such mouthpiece can be used without plasticization. In such case, the material to be used for the mouthpiece may be thermoplastic resin or the like.

The phrase “to the tooth form . . . or to a part thereof” used herein indicates that the scope of the present invention includes not only a mouthpiece that is applied to the entire tooth form of the upper or lower jaw, but also a mouthpiece that is applied to a part of such a tooth form. That is, the mouthpiece used in the present invention may fit a part of the tooth form such as teeth on the left side of the upper or lower jaw, or front teeth. Since a mouthpiece is applied to a patient subjected to treatment in a manner such that the shape of such mouthpiece corresponds to the tooth form of the patient, even a mouthpiece for a part of the tooth form can be securely applied. However, when a mouthpiece for a part of the tooth form is used, a holding means may be used so that a mouthpiece applied to teeth is not easily removed or displaced. Such holding means may be any means whereby a patient never or rarely feels discomfort such as pain, and whereby the mouthpiece can easily be removed after treatment; however, the holding means is not particularly limited thereto. Examples of such holding means include fastenings formed with metals or plastics, adhesives, and adhesive tapes.

Materials used for producing the mouthpiece of the present invention are not particularly limited, and any materials known in the field can be used. Preferably, such materials are excellent in terms of durability, formability, and shock absorption, and are not (or are only slightly) biohazardous, cost effective, and easily available. The material used may be any hard material (such as metal or rigid resin) or soft material (such as soft resin). In addition, the material may be transparent, translucent, or colored. The material can be adequately selected according to the type of a mouthpiece produced (stock type, mouth-formed type, custom-made type, or a combination thereof).

For instance, examples of a material having a relatively high softening point, which is used for a stock type or custom-made type mouthpiece, include vulcanized rubber and elastic plastic. More specifically, a rubber elastic body such as silicon rubber, ethylene-vinyl acetate (EVA) or polyolefin can be used. Preferably, thermosetting resin can be used.

In addition, as an example of thermoplastic resin having a low softening point, which is used for a mouth-formed type, a hot-melt material mainly consisting of a plastic elastic body, which becomes softened in a hot water between approximately 60° C. to 100° C., can be used. Examples of such hot-melt material include ethylene-vinyl acetate (EVA).

A mouthpiece (mouth-formed type) in which the body part and the tooth form part are formed with thermoplastic resin, is preferable in view of the ability of a patient to easily mold a mouthpiece himself or herself immediately before use in a manner such that the shape of the mouthpiece corresponds to his or her tooth form. Meanwhile, in the practice of dental medicine, preferably, a mouthpiece (custom-made type) formed with thermosetting resin is used.

The mouthpiece used in the present invention may be a disposable type mouthpiece, which is discarded after a single use, or a reusable type mouthpiece, which is collected after use and is reused following adequate washing or the like. Based on the adequate selection of the material used, or the like, either a disposable type or reusable type mouthpiece can be produced according to need.

The mouthpiece used in the present invention comprises an EL element. In the present invention, an EL element may be attached to a mouthpiece to be used. As an example of the present invention, an EL element can be removably applied to a mouthpiece. Specifically, an EL element can be incorporated into the lower layer or the sidewalls (facing the lip side and the cheek side in the oral cavity) of a mouthpiece. Alternatively, the mouthpiece may have a detachable double-layer structure so that an EL element is removably applied between the upper layer and the lower layer.

An EL element incorporated in or attached to a mouthpiece in the present invention may be any type that emits light, without particular limitation, but specifically an EL element that emits light having a wavelength of 300 nm to 1000 nm is preferred, and an EL element that emits visible light (for example, visible light having a wavelength of 400 nm to 700 nm) is more preferred. An EL element may have any shape that can be attached to a mouthpiece, without particular limitation, but an EL element is preferably a sheet which can be cut, and more preferably a sheet having a thickness of 2 mm or less.

An EL element emits light upon an application of an electrical field to a fluorescent compound contained therein, and an EL element can be categorized in an organic EL or an inorganic EL, depending on a type of the light emitting layer thereof. In the present invention, either an organic EL or an inorganic E may be used.

In the present invention, preferably an electroluminescence (EL) sheet is used. An EL sheet is a surface emitting device which emits light upon an application of an alternating voltage, and is characterized by that: it is thin and flexible; it consumes only a small amount of power; it can be easily cut by scissors or the like; and it does not generate heat. Specific examples of an EL sheet which may be used in the present invention include those commercially available from SEIKO Precision Inc., and Taiyo Kogyo Corp. An inorganic EL display and an organic EL display may be used for the present purpose.

The light irradiation device of the present invention is equipped with a power source for cause light to be emitted from an EL. The power source may be a battery cell (preferably, a small battery cell which can be attached to the inside of a mouthpiece), or a DC power source which may be converted from commercial AC power source and supplied via a power line.

The EL element preferably has a plastic layer laminated on the surface thereof. Examples of the plastic include, but are not limited to, polypropylene, polyethylene, polyethylene terephthalate, polyethylene styrene, polycarbonate, polyethersulfine, polyester, and vinyl chloride. The lamination of a plastic layer forms a mouth guard which allows medicine to be directly applied to an EL. Also, the lamination of a plastic layer makes it possible to form an EL/mouthpiece which is composed of only an EL along a target dentition.

At least one type of electroluminescence (EL) element is contained in or applied to a mouthpiece used in the present invention. Further, any supplemental drugs, including the following (1) to (3), may be used in combination: (1) dental treatment drugs; (2) disinfectants for affected areas; and (3) absorbents used for absorption of body fluids such as saliva, blood and pus. In addition, the mouthpiece used in the present invention is kept in a mouth for a certain period of time for dental disease treatment. During such period, body fluids such as saliva, blood and pus may be produced or effused in lesions. When such body fluids may negatively influence dental disease treatment, or when such body fluids undesirably cause a patient to feel discomfort, it is preferable that an absorbent be retained on a mouthpiece such that the body fluids are absorbed thereinto.

The duration for which the mouthpiece used in the present invention is worn is not particularly limited. However, preferably, the duration is to an extent such that a patient does not experience considerable inconvenience in his or her daily life, and at the same time, to an extent such that sufficient treatment effects can be achieved. As a specific example, the duration may range from minutes to hours (e.g., 1 minute to 10 hours), tens of hours (e.g., 10 hours to 24 hours), days (e.g., a day to 9 days), or tens of days (e.g., 10 days to 30 days or more). Thus, the duration can be freely determined.

Hereafter, the shape of a mouthpiece and usage thereof will be described more specifically with reference to FIG. 5. Note that the mouthpiece described in FIG. 5 merely indicates one embodiment of the present invention, and the mouthpiece is not limited to the shape described in FIG. 5.

Preferably, mouthpiece 1 is formed with thermosetting resin or thermoplastic resin, in which groove 2 is formed along with the tooth form. On the one side of groove 2, sidewall 3 that faces the jaw side and the lip side in the oral cavity is formed, and on the other side thereof, sidewall 4 that faces the palate side in the oral cavity is formed. In addition, mouthpiece 1 can be produced by adequately adjusting the heights and lengths extending to edge 5, of sidewalls 3 and 4, depending on the lengths of teeth and the gum of a user. Or, mouthpiece 1 can be cut using scissors or the like immediately before use.

When mouthpiece 1 is formed with thermoplastic resin, a container filled with hot water is previously prepared and mouthpiece 1 is soaked with such hot water. After mouthpiece 1 becomes plasticized, the tooth form is made on the mouthpiece by applying it to teeth of the upper or lower jaw and the gum of a patient and having the patient bite it softly or press it with fingers.

Then, the mouthpiece on which the tooth form has been made is removed from the mouth, an electroluminescence (EL) element is applied to the inside of groove 2 (that is, where teeth and/or the gum of the patient come into contact with the mouthpiece), and then the mouthpiece is applied again to the teeth and the gum. In addition, when using the mouthpiece of the present invention, it is preferable that toothbrushing is previously carried out using a toothbrush such that mouthpiece 1 can tightly come into contact with teeth and the gum.

After the elapse of a given time for wearing mouthpiece 1, the mouthpiece is removed from the jaw so that the treatment can be terminated. After use, mouthpiece 1 (in the case of reusable type) is cleanly washed with warm water and a wash or the like, and thus it can be stored before reuse.

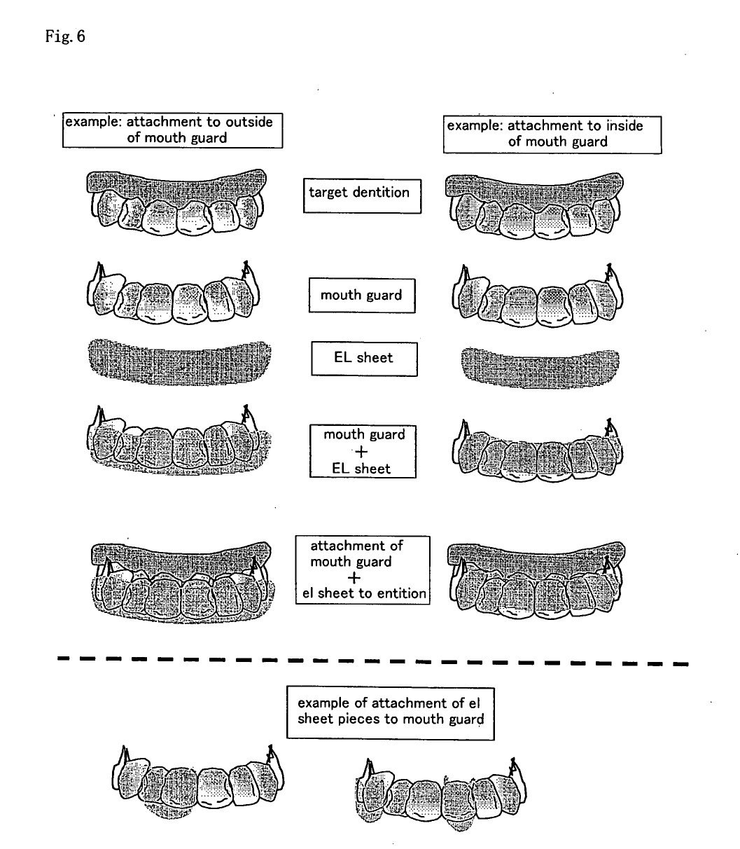

FIG. 6 shows an attachment of an EL sheet to a mouthpiece (mouth guard). An EL sheet may be attached to the outside of a mouth guard (the side opposite to the tooth surface), or to the inside of the mouth guard (the side facing to the tooth surface). When an EL sheet is attached to the outside of a mouth guard, the light emitted by the EL sheet passes through the mouth guard to reach the tooth surface.

The present invention will be more specifically explained below by way of Examples, but the present invention will not be limited to these Examples.

### EXAMPLE

In each of the following Examples 1 to 4, EL sheets of high brightness type (manufactured by Seiko Precision Inc.) 130×120 mm were used.

### Example 1

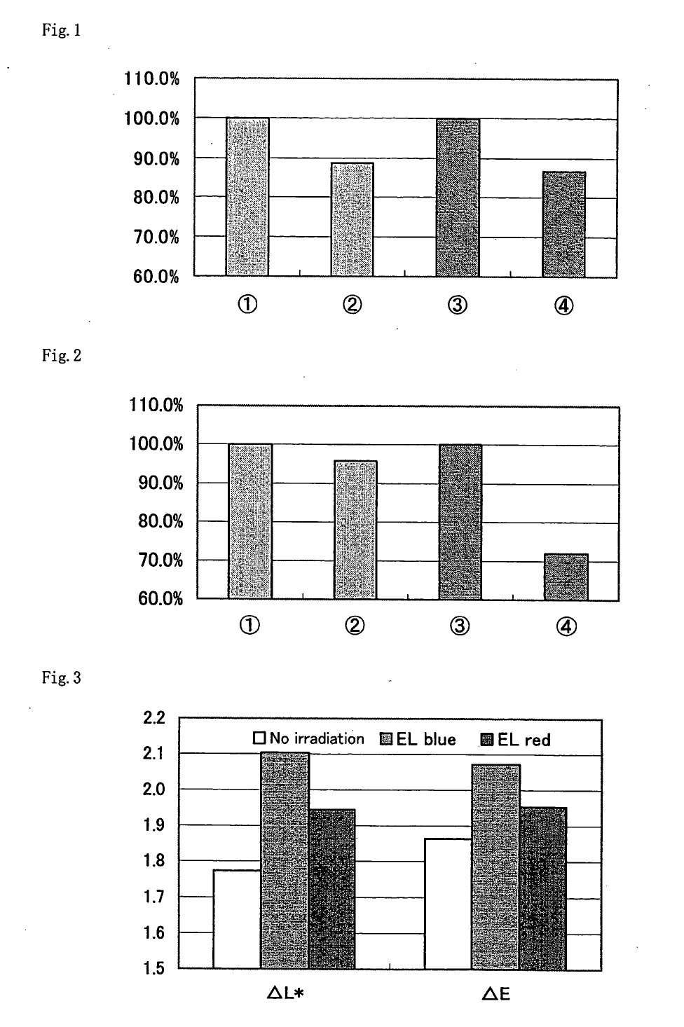

Sample liquids from No. 1 to No. 4 were added to a discolored 5,000 ppm tetracycline hydrochloride solution (5 mL). Samples No. 1 and No. 3 were heated at a temperature of 37° C. for 15 minutes and 40 seconds in a dark room, while Samples No. 2 and No. 4 were irradiated by a yellow EL sheet (manufactured by Seiko Precision Inc.) from the above for 40 seconds, and then were heated at a temperature of 37° C. for 15 minutes. Each Sample were centrifuged at a speed of 8,000 rpm for 5 minutes to obtain a supernatant liquid, and an absorbance of the supernatant liquid was measured using a wavelength of 358 nm, that is, the wavelength at which maximum absorption of tetracycline hydrochloride occurs. In each Sample, Samples No. 1 and No. 2 were 35% hydrogen peroxide (5 mL) and Samples No. 3 and No. 4 were titanium oxide (1 g) and distilled water (5 mL). The results are shown in FIG. 1. In FIG. 1, the absorbance of Sample No. 1 was set to be 100, and the absorbance of Sample No. 3 was set to be 100.

### Example 2

Sample liquids from No. 1 to No. 4 were added to a discolored 0.1% methylene blue solution (5 mL). Samples No. 1 and No. 3 were heated at a temperature of 37° C. for 15 minutes in a dark room, while Samples No. 2 and No. 4 were irradiated by a green EL sheet (manufactured by Seiko Precision Inc.) from the above for 40 seconds, and then were heated at a temperature of 37° C. for 15 minutes. Each Sample were centrifuged at a speed of 8,000 rpm for 5 minutes to obtain a supernatant liquid, and an absorbance of the supernatant liquid was measured using a wavelength of 665 nm, that is, the wavelength at which maximum absorption of methylene blue occurs. In each Sample, Samples No. 1 and No. 2 were 35% hydrogen peroxide (5 mL), and Samples No. 3 and No. 4 were titanium oxide (1 g) and distilled water (5 mL). The results are shown in FIG. 2. In FIG. 2, the absorbance of Sample No. 1 was set to be 100, and the absorbance of Sample No. 3 was set to be 100.

### Example 3

A filter paper having a diameter of 6 mm was immersed in a 0.1% hematoporphyrin solution, and was dried, and then the initial color of the resulting paper was measured by a colorimeter (E1). The filter paper and 35% hydrogen peroxide (8 mL) were put in a plastic dish having a diameter of 50 mm, which was irradiated by a red EL sheet (manufactured by Seiko Precision Inc.) and a blue EL sheet (manufactured by Seiko Precision Inc.) for 40 seconds, and then heated at a temperature of 37° C. for 15 minutes. For a non-illuminated blank, the dish was covered with aluminum foil, and then heated at a temperature of 37° C. for 15 minutes and 40 seconds. After the heating, the filter paper was washed with distilled water (40 mL) and dried, and the color was measured by a colorimeter (colormate) (E2). The results are shown in FIG. 3. FIG. 3 shows ΔL and ΔE which are obtained by subtracting the initial color values (E1) from the color values changed after light irradiation (E2).

### Example 4

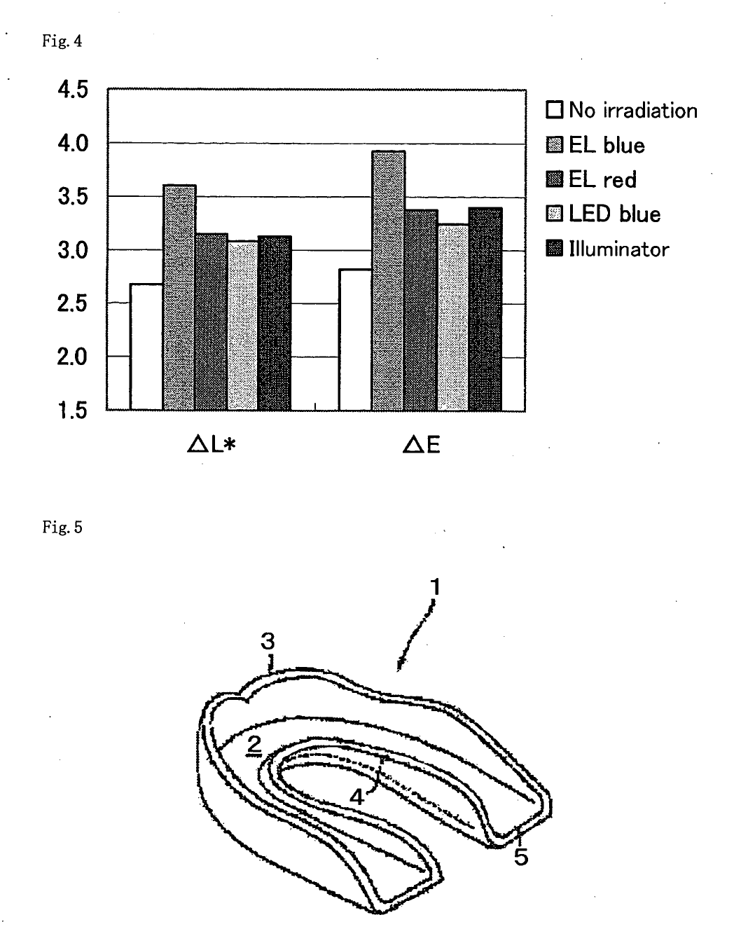

A filter paper having a diameter of 6 mm was immersed in a 0.1% hematoporphyrin solution, and was dried, and then the initial color of the resulting paper was measured by a calorimeter (E1). Into a plastic dish having a diameter of 50 mm, distilled water (8 mL), titanium oxide (0.2 g), and the above filter paper were put, which were illuminated by a red EL sheet (manufactured by Seiko Precision Inc.), a blue EL sheet (manufactured by Seiko Precision Inc.), a blue LED, and a dental illuminator for 40 seconds, and then were heated at a temperature of 37° C. for 15 minutes. For a non-illuminated blank, the dish was covered with aluminum foil, and then heated at a temperature of 37° C. for 15 minutes and 40 seconds. After the heating, the filter paper was washed with distilled water (40 mL) and dried, and the color was measured by a calorimeter (colormate) (E2). The results are shown in FIG. 4. FIG. 4 shows ΔL and ΔE which are obtained by subtracting the initial color values (E1) from the color values changed after light irradiation (E2).

### INDUSTRIAL APPLICABILITY

As described above, the present invention provides means which makes it possible to irradiate the oral cavity in a simple and safe way without heat generation.

1. An intraoral light irradiation device, wherein an electroluminescence (EL) element is used.

2. The intraoral light irradiation device according to claim 1, which is composed of a mouthpiece having a electroluminescence (EL) element.

3. The intraoral light irradiation device according to claim 1, wherein the electroluminescence (EL) element can emit visible light having a wavelength from 300 nm to 1000 nm.

4. The intraoral light irradiation device according to claim 1, wherein the electroluminescence (EL) can emit visible light.

5. The intraoral light irradiation device according to claim 4, wherein the electroluminescence (EL) element can emit blue or green visible light.

6. The intraoral light irradiation device according to claim 1 to 5, wherein the electroluminescence (EL) element is formed in a sheet.

7. The intraoral light irradiation device according to claim 6, wherein the electroluminescence (EL) element is formed in a sheet having a thickness of 2 mm or less.

8. The intraoral light irradiation device according to claim 1, wherein the electroluminescence (EL) element is an organic EL.

9. The intraoral light irradiation device according to claim 1, wherein a display-type EL element which is formed by depositing electroluminescence (EL) on a glass substrate and illuminates upon an application of voltage, is used.

10. The intraoral light irradiation device according to claim 1, wherein the plastic is laminated on the surface of the electroluminescence (EL) element.

11. The intraoral light irradiation device according to claim 1, wherein the mouthpiece has a concavo-convex shape which is adapted to a dentition, and the concavo-convex shape is provided with the electroluminescence (EL) element.
